(19) United States
(12) Patent Application Publication
(10) Pub. No.: US 2019/0167561 A1
(43) Pub. Date: Jun. 6, 2019

(54) METHODS AND COMPOSITIONS FOR REDUCING THE GRAYING OF HAIR

(71) Applicants: Natura Cosmeticos SA, Sao Paulo (BR); The General Hospital Corporation, Boston, MA (US)

(72) Inventors: Ana Paula Pedroso Oliveira, Sao Paulo (BR); Gabriela Placoná Diniz, Sao Paulo (BR); Juliana Carvalhães Lago, Sao Paulo (BR); Kelen Fabiola Arroteia, Sao Paulo (BR); David E. Fisher, Newton, MA (US)

(21) Appl. No.: 16/325,506

(22) PCT Filed: Aug. 11, 2017
(86) PCT No.: PCT/US17/46541

§ 371 Date: Feb. 14, 2019

Related U.S. Application Data
(60) Provisional application No. 62/375,286, filed on Aug. 15, 2016.

(51) Int. Cl. A61K 8/9789; A61K 8/9728; A61K 8/49; A61K 8/64; A61K 8/67; A61K 8/44; A61Q 5/10

(52) U.S. Cl. CPC A61K 8/9789; A61K 8/9728; A61K 8/498; A61K 8/645; A61K 2800/522; A61K 8/447; A61Q 5/10; A61K 2800/5922; A61K 8/678

(57) ABSTRACT

This disclosure relates to methods of reducing or inhibiting the graying of hair by topically applying various cosmetic formulations to hair follicles and/or to the skin overlying hair follicles. The formulations applied in these methods include green tea extract and one or more additional plant extracts, in addition to one or more cosmetically acceptable carriers and/or accessory ingredients.

The subject matter disclosed in U.S. Patent Application No. 62/129,189 was developed and the claimed invention described herein were made by, or on behalf of, parties to a Joint Research Agreement (“JRA”) under AIA 35 U.S.C. 102(c) that was in effect on or before the effective filing date of the invention claimed in the present application; the invention claimed herein was made as a result of activities undertaken within the scope of the JRA; and the names of the parties to the JRA are Partners Healthcare and Natura Cosméticos S.A.

### TECHNICAL FIELD

This invention relates to methods for reducing the graying of hair.

### BACKGROUND

The hair-graying process represents a prominent feature of aging. Previous studies have utilized a mouse model to examine hair graying (Nishimura et al, Science 2005). In this model, the melanocyte lineage was “tagged” by use of a marker (beta-galactosidase, or LacZ), which permitted identification of rare melanocyte stem cells within the bulge region of the hair follicle.

### SUMMARY

The present disclosure is based, at least in part, on the discovery that the cosmetic formulations described herein are useful in reducing the graying of hair, as well as new compositions and new assay methods to determine the effectiveness of new compositions to inhibit the graying of hair. As described herein, the graying of hair can be reduced or inhibited by topically applying to the scalp or hair follicles compositions and formulations comprising green tea extract and other plant extracts in cosmetically effective amounts to prevent or reduce hair follicle melanocyte stem cell damage and differentiation into pigmented cells. The formulations used in the new methods provided herein comprise green tea extract, one or more additional plant extracts, and a cosmetically acceptable carrier and/or accessory ingredients.

In one aspect, the disclosure provides for a method for reducing or inhibiting the graying of hair, the method comprising topically applying a cosmetically effective amount of a formulation to hair follicles and/or to the skin overlying hair follicles one or more times per day, wherein the formulation comprises a green tea extract; one or more of the group consisting of aroeira (Schinus terebinthifolius) extract, coffee extract, vitamin E, grape seed oligomeric proanthocyanidins (OPC), cocoa extract, Castanea sativa seed extract, hydrolyzed Candida saitoana extract and soybean protein; and one or more cosmetically acceptable carriers comprising an aqueous gel, alcoholic gel, ointment, oil, alcoholic or aqueous fluid, water-in-oil emulsion, oil-in-water emulsion, and a water-in-silicone emulsion.

In some embodiments, the formulation further comprises one or more cosmetically acceptable accessory ingredients. In some embodiments, the cosmetically acceptable accessory ingredients are selected from the group consisting of xanthan gum, glycerine, EDTA, sodium benzoate, phenoxyethanol, 2-hydroxy fatty alcohol alkoxylate, sodium polyacrylate, polysorbate 20, BHT, disaccharidic gums, ethylhexylglycerin, carbomer, butyleneglycol, acrylate polymers, PEG-40 hydrogenated castor oil, methylisothiazolinone, methylchloroisothiazolinone, propylene glycol, potassium sorbate, polyglyceryl caprylate, fragrance and water. In some embodiments, the combination comprises between about 0.1% and 15% of the formulation.

In some embodiments, the green tea extract comprises about 0.001% to about 0.5% of the formulation. In some embodiments, the green tea extract comprises about 0.08% to about 0.2% of the formulation. In some embodiments, aroeira (Schinus terebinthifolius) extract is included and the aroeira (Schinus terebinthifolius) extract comprises about 0.001% to about 2.5% of the formulation. In some embodiments, aroeira (Schinus terebinthifolius) extract is included and the aroeira (Schinus terebinthifolius) extract comprises about 0.08% to about 0.5% of the formulation. In some embodiments, coffee extract is included and the coffee extract comprises about 0.01% to about 1% of the formulation. In some embodiments, coffee extract is included and the coffee extract comprises about 0.08% to about 0.1% of the formulation. In some embodiments, vitamin E is included and the vitamin E comprises about 0.01% to about 3% of the formulation. In some embodiments, vitamin E is included and the vitamin E comprises about 0.08% to about 1% of the formulation. In some embodiments, grape seed oligomeric proanthocyanidins (OPC) is included and the grape seed oligomeric proanthocyanidins (OPC) comprises about 0.01% to about 1% of the formulation. In some embodiments, grape seed oligomeric proanthocyanidins (OPC) is included and the grape seed oligomeric proanthocyanidins (OPC) comprises about 0.08% to about 0.1% of the formulation. In some embodiments, cocoa extract is included and the cocoa extract comprises about 0.01% to about 1% of the formulation. In some embodiments, cocoa extract is included and the cocoa extract comprises about 0.05% of the formulation. In some embodiments, Castanea sativa seed extract is included and the Castanea sativa seed extract comprises about 0.2% to about 5% of the formulation. In some embodiments, Castanea sativa seed extract is included and the Castanea sativa seed extract comprises about 1% to about 3% of the formulation. In some embodiments, hydrolyzed Candida saitoana extract is included and the hydrolyzed Candida saitoana extract comprises about 0.08% to about 3% of the formulation. In some embodiments, hydrolyzed Candida saitoana extract is included and the hydrolyzed Candida saitoana extract comprises about 0.1% to about 3% of the formulation. In some embodiments, hydrolyzed Candida saitoana extract is included and the hydrolyzed Candida saitoana extract comprises about 0.08% to about 2% of the formulation. In some embodiments, soybean protein is included and the soybean protein comprises about 0.5% to about 15% of the formulation. In some embodiments, soybean protein is included and the soybean protein comprises about 0.1% to about 6% of the formulation.

In some embodiments, xanthan gum is included and the xanthan gum comprises about 0.001% to about 5% of the formulation. In some embodiments, glycerin is included and the glycerin comprises about 0.001% to about 5% of the formulation. In some embodiments, EDTA is included and the EDTA comprises about 0.001% to about 5% of the formulation. In some embodiments, sodium benzoate is included and the sodium benzoate comprises about 0.001% to about 5% of the formulation. In some embodiments, phenoxyethanol is included and the phenoxyethanol comprises about 0.001% to about 5% of the formulation. In some embodiments, 2-hydroxy fatty alcohol alkoxylate is included and the 2-hydroxy fatty alcohol alkoxylate comprises about 0.001% to about 5% of the formulation. In some embodiments, sodium polyacrylate is included and the sodium polyacrylate comprises about 0.001% to about 5% of the formulation. In some embodiments, polysorbate 20 is included and the polysorbate 20 comprises about 0.001% to about 5% of the formulation. In some embodiments, BHT is included and the BHT comprises about 0.001% to about 5% of the formulation. In some embodiments, fragrance is included and the fragrance comprises about 0.001% to about 5% of the formulation. In some embodiments, water is included and the water comprises about 0.001% to about 5% of the formulation. In some embodiments, the formulation comprises about 0.1% of green tea extract, about 0.25% of aroeira (Schinus terebinthifolius) extract and about 0.1% of grape seed oligomeric proanthocyanidins (OPC).

In some embodiments, the formulation comprises about 2.2% of Castanea sativa seed extract, about 1.65% of hydrolyzed Candida saitoana extract, about 0.05% of cocoa extract and about 5.5% soy protein. In some embodiments, the formulation further comprises N-acetyl-cysteine.

In some embodiments, the formulation reduces oxidative cell stress. In some embodiments, the formulation inhibits melanocyte stem cell depletion.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

FIG. 1 is a schematic illustration of an assay method protocol used to identify formulations or active agents useful in reducing or inhibiting the graying of hair.

FIG. 2 is a bar graph of the pigmented cell count in hair treated with various controls (CPC=Positive Control) and with Formulation A (Sample A).

FIG. 3 is a graph of the pigmentation efficiency in hair treated with various controls (CPC=Positive Control) and with Formulation A (Sample A).

FIG. 4 is a graph of the pigmented cell count in hair treated with various controls (CPC=Positive Control) and with Formulation B (Sample B).

FIG. 5 is a graph of the pigmentation efficiency in hair treated with various controls (CPC=Positive Control) and with Formulation B (Sample B).

FIG. 6 is a graph of the pigmented cell count in hair treated with various controls (CPC=Positive Control) and with Formulation C (Sample C).

FIG. 7 is a graph of the pigmentation efficiency in hair treated with various controls (CPC=Positive Control) and with Formulation C (Sample C).

FIG. 8 is a graph of the pigmented cell count in hair treated with various controls (CPC=Positive Control) and with Formulation D (Sample D).

FIG. 9 is a graph of the pigmentation efficiency in hair treated with various controls (CPC=Positive Control) and with Formulation D (Sample D).

FIG. 10 is a series of representations of photos of the ectopic pigmentation in a bulge area of the follicles after no treatment, and treatment with H₂O₂ alone (which induces oxidative damage), with Positive Control (CPC) plus H₂O₂, wherein the CPC was N-acetyl-cysteine (NAC), with Placebo A, with Placebo B, C, and D, and with Formulations A, B, C, and D.

FIG. 11 is a series of photographic images and a bar graph showing the results of an ANOVA test measuring the Reactive Oxygen Species (ROS) decrease with N-acetyl-cysteine (NAC) treatment.

FIG. 12 is a graph of pigmented cell counts with N-acetyl-cysteine treatment and other control and stress-inducing treatments.

FIGS. 13A, 13B, 13C, and 13D are representations of photos of pigmented melanoblasts in bulge areas on hair follicles. FIG. 13A and FIG. 13C are images of melanoblasts before culturing and FIG. 13B and FIG. 13D are melanoblasts after 7 days culture. FIG. 13A and FIG. 13B were not treated with genotoxic stress; FIG. 13C and FIG. 13D were treated with genotoxic stress. Circles in FIG. 13D indicate pigments.

FIG. 14 is a bar graph illustrating the pigments per follicle after topically applying a blank control, hydrogen peroxide (H₂O₂), a positive control (CPC) with H₂O₂, Formulation A with H₂O₂, and placebo with H₂O₂.

### DETAILED DESCRIPTION

Provided herein are methods for reducing the graying of hair by applying cosmetically effective amounts of topical compositions to the hair follicles, e.g., to the scalp and/or the hair. The compositions (or formulations) include a green tea extract as a first active agent and one, two, three, four, five, six, seven, or all eight of the following active agents: aroeira (Schinus terebinthifolius) extract, grape oligomeric proanthocyanidins (OPC), cocoa extract, coffee extract, vitamin E, sweet chestnut (Castanea sativa) seed extract (Recoverine®), hydrolyzed Candida saitoana extract (Celldetox®), and soy protein (Glycine Soja), as well as one or more cosmetically acceptable carriers to form, for example, a cream, lotion, or shampoo. In some embodiments, the composition further includes one or more additional plant extracts and cosmetically acceptable carriers and/or accessory ingredients.

### Methods of Inhibiting Graying of Hair

The methods of inhibiting or reducing graying of hair described herein include reducing graying of hair as a result of age and/or stress, for example oxidative stress, e.g., as caused by hair treatments that include bleaching the hair. In some embodiments the methods provide for reducing or inhibiting the graying of hair as a result of genotoxic stress as a result of oxidative agents such as hydrogen peroxide (H₂O₂). Thus, the methods described herein include applying the compositions before or after, e.g., directly before or after, a hair treatment such as bleaching or dying the hair. Described herein are methods for reducing or inhibiting the graying of hair by reducing the oxidative damage to the melanocyte stem cells (MSCs) and thereby reducing the differentiation and pigmentation of melanocytes, which leads to irreversible hair graying.

In some embodiments, the formulations applied in the methods described herein include about 0.1% to about 15% of a combination of green tea extract and one or more additional plant extracts. For example, the formulation may include about 0.01 to 0.5% (e.g., about 0.1%, or about 0.08 to 0.2%) green tea extract in combination with about 0.01 to about 2.5% (e.g., about 0.25%, or about 0.08 to 0.5%) aroeira (Schinus terebinthifolius) extract, and/or about 0.01 to about 1% (e.g., about 0.1%, or 0.08 to 0.1%) grape seed oligomeric proanthocyanidins (OPC), and/or about 0.01 to about 1% (e.g., about 0.08 to 0.1%) coffee extract, and/or about 0.01 to about 3% (e.g., about 0.08 to 1%) vitamin E and/or about 0.01 to about 1% (e.g., about 0.05%, or about 0.08 to 1%) cocoa extract, and/or about 0.2 to about 5% (e.g., about 2.2%, about 2%, or about 1 to 3%,) Castanea sativa seed extract (Recoverine®), and/or about 0.08 to about 3% (e.g., about 1.65%, about 1% to about 2%, or about 0.08 to 2%) hydrolyzed Candida saitoana extract (Celldetox®) and/or about 0.5 to about 15% (e.g., about 5.5%, about 0.1 to 6%, or about 1 to 6%) soy protein.

In some cases, the extracts described herein are obtained by extraction processes known in the art, see for example WO2015/031971 which is incorporated herein in its entirety.

The formulations used in the methods described herein can also include one or more cosmetically acceptable carriers and/or cosmetically acceptable accessory ingredients. Suitable cosmetically acceptable carriers include, but are not limited to, aqueous gels, alcoholic gels, ointments, oils, alcoholic or aqueous fluids, water in oil emulsions, oil in water emulsions, and water in silicone emulsions. Suitable cosmetically acceptable accessory ingredients include, but are not limited to, xanthan gum, glycerin, EDTA, sodium benzoate, phenoxyethanol, 2-hydroxy fatty alcohol alkoxylate, sodium polyacrylate, polysorbate 20, BHT, disaccharidic gums, ethylhexylglycerin, carbomer, butyleneglycol, acrylate polymers, PEG-40 hydrogenated castor oil, methylisothiazolinone, methylchloroisothiazolinone, propylene glycol, potassium sorbate, polyglyceryl caprylate, fragrance, and water.

In some embodiments of the methods described herein the applied formulations comprise from 0 to about 5% of xanthan gum, about 0.1 to about 3% of glycerin, about 0.01 to about 0.1% of EDTA, about 0.1 to about 1.0% of sodium benzoate, about 0.1 to about 2% of phenoxyethanol, about 0.1 to about 3% of 2-hydroxy fatty alcohol alkoxylate, about 0 to about 2% of sodium polyacrylate, about 0 to about 4% of polysorbate 20, about 0.01 to about 0.1 of BHT, 0 to about 1% of fragrance and from about 80 to about 98% of water, preferably demineralized water.

In general, the active agents in the formulations used in the methods described herein have antioxidative or antioxidant activity. As described herein an antioxidative activity of an agent can be determined using chemiluminescence and measuring the induced chemiluminescence of human skin (ICL-S). The ICL-S signal is detected by a photomultiplier tube (PMT), providing a continuous and non-invasive monitoring of oxidative stress in skin in vivo following the application of cosmetic products. Other methods of measuring antioxidant activity are known in the art.

Thus, the formulations used for the methods described herein can include one or more antioxidants. The antioxidant can be, for example, a small molecule compound, a peptide, or a protein. In some embodiments the antioxidant contains a thiol group or is a derivative of the amino-acid cysteine. A non-limiting list of antioxidants includes N-acetyl-cysteine (NAC), Vitamin C, and Vitamin E. In some embodiments, the formulation includes NAC.

In general, the methods of reducing or inhibiting graying of hair include the application of a cosmetically effective amount of the formulation to hair follicles and/or to the skin overlying hair follicles. A “cosmetically effective amount” is an amount sufficient to effect beneficial or desired results in reducing the ongoing graying of hair. This amount can be the same or different from a “prophylactically effective amount,” which is an amount necessary to prevent or inhibit the onset of gray hair. An effective amount can be administered in one or more administrations, applications or dosages. A cosmetically or prophylactically effective amount of a formulation (i.e., an effective dosage) depends on the specific anti-graying formulation selected. The anti-graying compositions can be administered from one or more times per day to one or more times per week; including one to five times per day, e.g., once, twice, or three times every day, or one to five times every other day, e.g., once, twice, or three times every other day. The skilled artisan will appreciate that certain factors may influence the dosage and timing required to effectively treat a subject, including but not limited to the severity of the gray hair, previous treatments, the general health and/or age of the subject, and any diseases present. Moreover, treatment of a subject with a cosmetically or prophylactically effective amount of the cosmetic formulations and compositions described herein can include a single treatment or a series of treatments.

Dosage, toxicity, and cosmetic or prophylactic efficacy of the formulations can be determined by standard procedures in cell cultures or experimental animals, e.g., for determining the ED50 (the dose cosmetically effective in 50% of the population). The data obtained from cell culture assays and animal studies can be used in formulating a range of dosage for use in humans. The dosage may vary within this range depending upon the dosage form employed and the route of administration utilized. For any formulation or composition used in the methods of reducing the graying of hair described herein, the cosmetically or prophylactically effective dose can be estimated initially from cell culture assays.

The methods of reducing graying of hair described herein can also include topically administering an additional cosmetically active ingredient that can be administered before, after, or concurrently with the green tea extract formulation.

The topical cosmetic formulations of the methods described herein can be formulated in compositions for different types of administration, for example, the formulation may be formulated into a composition for application as a spray, a cream, a lotion, an ointment, in an emulsion, a shampoo, a hair gel, a hair dye, a hair tonic, an oil, a liquid, a emollient, a moisturizer, a sunscreen, and can be an alcohol-free or an alcohol-based liquid, gel, emulsion, cream, etc.

In some embodiments, the methods provided herein reduce the oxidative cell stress and/or damage in the hair and/or hair follicles. In some embodiments, the methods provided herein reduce melanocyte stem cell depletion. In some embodiments, the methods provided herein reduce differentiation of melanocyte stem cells into pigmented cells.

### Assay Methods

This disclosure further provides assays for screening potential active agents that reduce graying of hair. The assay methods described herein include inducing oxidative stress, e.g., by the application of an H₂O₂ solution or ionizing radiation, and topically administering the test agents to human primary hair follicles and detecting one or more of a reduction in oxidative damage, a reduction in differentiation of the melanocyte stem cell differentiation into pigmented cells, and a reduction in the graying of the hair. In general, the primary hair follicles are grown and maintained in culture and the oxidative stress can be determined before, during, and after treatments with the test agent. Untreated hair follicles and hair follicles to which a standard antioxidant is applied can be used as a negative and a positive control reference, respectively.

In some embodiments, the assay methods of screening for agents that reduce or inhibit graying of hair include isolating follicles from a human specimen, embedding them in a semisolid media, and topically administering the oxidative stress-inducing agent and the test active agent, in any order or simultaneously, or at different time intervals between administrations, e.g., once daily, twice daily, or 5, 10, 15, 20 or more minutes apart, or 1, 2, 3, 4, 5 or more hours apart, or 1, 2, 3, 4 or more days apart. The assay methods described herein can further comprise detecting or imaging pigmented cells in the specimen or measuring the level of oxidative activity in comparison to a reference specimen. Methods for detecting or imaging cells, and measuring levels of oxidative activity are known in the art.

Included herein are methods for screening agents (test molecules), e.g., plant extracts, polypeptides, polynucleotides, or inorganic or organic large or small molecule compounds, to identify agents useful in reducing hair graying. As used herein, “small molecules” refers to small organic or inorganic molecules of molecular weight below about 3,000 Daltons. In general, small molecules useful for the invention have a molecular weight of less than 3,000 Daltons (Da).

Test compounds and mixtures identified as hits can be considered for cosmetically or prophylactically effective formulations, useful in reducing or inhibiting the graying of hair. A variety of techniques useful for determining the structures of “hits” can be used in the methods described herein, e.g., NMR, mass spectrometry, chromatography equipped with electron capture detectors, fluorescence and absorption spectroscopy. Thus, the invention also includes compounds identified as “hits” by the methods described herein, and methods for their administration and use in the treatment, prevention, or delay of development or progression of the graying of hair.

### EXAMPLES

The invention is further described in the following examples, which do not limit the scope of the invention described in the claims.

### Example 1: Formulations for Applying to Hair Follicles to Reduce Graying

A formulation for reducing the graying of hair was produced by adding phases 1-8 shown in Table 1 below in sequence. The ninth phase was prepared by mixing solubilizer, fragrance, and BHT, stirring the mixture and then adding to the main mixture. In phase 10, xanthan gum was slowly added until complete dispersion and the pH was adjusted to between about 5 and 6.

TABLE 1

| Phase | Component | Concentration (%) | Amount (g) |
|---|---|---|---|
| 1 | Demineralized water | 94.1000 | 94.1000 |
| 10 | Xanthan gum | 1.0000 | 1.0000 |
| 2 | Disodium EDTA | 0.0500 | 0.0500 |
| 3 | Sodium benzoate | 0.3000 | 0.3000 |
| 4 | Phenoxyethanol F | 0.8000 | 0.8000 |
| 5 | Aroeira extract | 0.2500 | 0.2500 |
| 6 | Grape seed OPC | 0.1000 | 0.1000 |
| 7 | Dry refined green tea extract | 0.1000 | 0.1000 |
| 8 | Glycerine | 1.5000 | 1.5000 |
| 9 | 2-hydroxy fatty alcohol | 1.5000 | 1.5000 |
| | alkoxylate | | |
| 9 | BHT | 0.0500 | 0.0500 |
| 9 | Fragrance | 0.2500 | 0.2500 |

Another formulation for applying to hair follicles to reduce the graying of hair was produced by adding phases 1-10, shown in Table 2 below, according to the indicated sequence. In phase 11, xanthan gum was slowly added until complete dispersion. Phase 12 was prepared by mixing solubilizer, fragrance, and BHT and adding that mixture to the main mixture. The pH was adjusted to 5-6.

TABLE 2

| Phase | Component | Concentration (%) | Amount (g) |
|---|---|---|---|
| 3 | Recoverine EL | 2.2000 | 2.2000 |
| 4 | Celldetox | 1.6500 | 1.6500 |
| 5 | Sodium Benzoate | 0.3000 | 0.3000 |
| 6 | Phenoxyethanol | 0.8000 | 0.8000 |
| 7 | Glycerine | 1.5000 | 1.5000 |
| 8 | Dry cacao extract | 0.0500 | 0.0500 |
| 9 | Soybean protein | 5.5000 | 5.5000 |
| 10 | Dry refined green tea extract | 0.1000 | 0.1000 |
| 1 | Demineralised water | 85.0500 | 85.0500 |
| 2 | Disodium EDTA | 1.0000 | 1.0000 |
| 11 | Xanthan gum | 0.0500 | 0.0500 |
| 12 | BHT | 0.2500 | 0.2500 |
| 12 | Fragrance | 1.5000 | 15000 |
| 12 | 2-hydroxy fatty alcohol | 0.1500 | 0.1500 |
| | alkoxylate | | |

Placebos A-D are the same as Formulations A-D without the active ingredients, e.g., without aroeira (Schinus terebinthifolius) extract, grape oligomeric proanthocyanidins (OPC), cocoa extract, coffee extract, vitamin E, sweet chestnut (Castanea sativa) seed extract (Recoverine®), hydrolyzed Candida saitoana extract (Celldetox®), or soy protein (Glycine Soja).

### Example 2: Pigmentation Assay

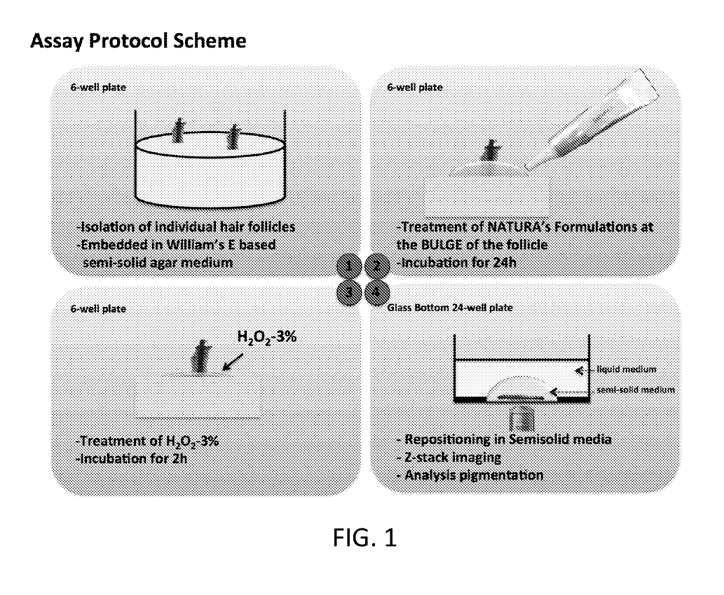

A novel platform for assaying human primary hair follicles in culture was developed to profile anti-graying potentially active agents (see FIG. 1). Normal human scalp specimens were obtained from various surgeries or from donor and recipient scalp hair derived from plastic surgery. Briefly, small scalp specimens (approximately 1 cm²) were transected around hairs by scalpel to remove unnecessary tissues and revealing all portions of the hair follicles embedded in the adipose tissue. Intact human skin specimens were implanted in semi-solid agarose (2% agarose diluted with Williams' medium E (Gibco™) at 1:5 ratio).

The hair follicle culture medium was based on Williams' medium E (Gibco™) supplemented with 2 mmol/L L-glutamine, hydrocortisone 10 ng/ml, insulin 10 μg/ml, penicillin 100 U/ml, streptomycin 100 μg/ml, and amphotericin B 25 μg/ml.

To maximize the effects of the test formulations and to exclude the variability of delivery ability of the formulations through the epidermis, the test formulations were applied directly at the bulge area of the follicles using the method as follows:

(1) tissue preparation and culture for 8-24 hrs.

(2) Isolation of individual follicles

(3) Vertically embedding in semisolid media

(4) Treatment with test formulation/Positive control on the bulge area of the hair follicles

(5) Incubation for 24 hrs.

(6) 3% H₂O₂ treatment

(7) Incubation for 2 hrs.

(8) Repositioning in semisolid media

(9) Z-stacking imaging & Analysis on day 1, 3, 7 after H₂O₂ treatment

For the experiment, a total of 602 follicles from 9 different donors were utilized. With small sized scalp tissue, the experiment was performed to validate one formulation as more than 8 follicles per each group were needed. With big sized scalp tissue, the experiment was performed to validate all the formulation A, B, C and D with the same donor tissue.

### Example 3: Pigmentation Assay with Formula A

With the protocol and donor tissues described above in Example 2, the assay was performed to observe the effectiveness of Formula A on melanocyte stem cell differentiation after stress of H₂O₂.

A total of 354 follicles from 7 different donors were used to validate the pigmentation efficiency of Formulation A. For the pigmented cell count, out of 7 donors, 6 donors with 260 follicles were used.

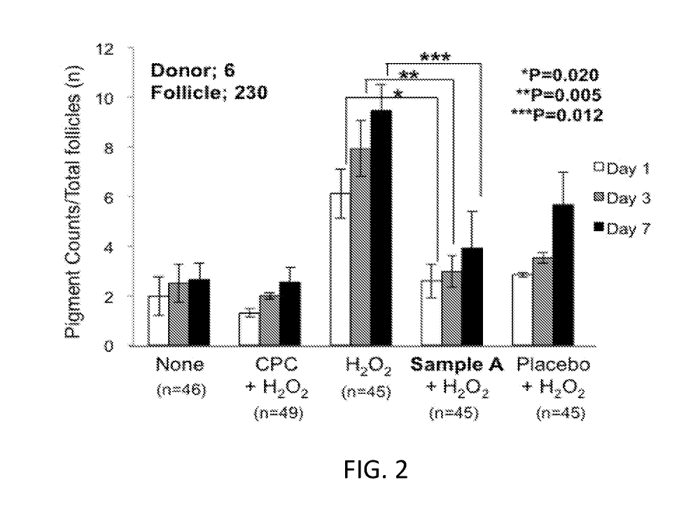

H₂O₂ treatment (3 percent) increased pigmentation at the bulge area of the follicle with statistical significance. Also the Positive Control (CPC), N-acetyl-cysteine (NAC), was effective on the pigmentation prevention with statistical significance. Pretreatment with Formulation A induced statistically significant decreased pigmented cell counts at the day 1, 3, 7 of H₂O₂ treatment compared to H₂O₂ alone, which implicates Formulation A has an effect on preventing melanocyte stem cell depletion (FIG. 2).

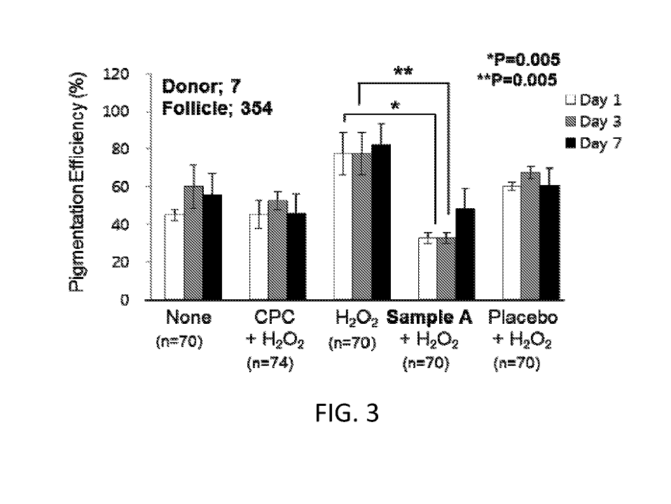

Pigmentation efficiency represents pigmented follicle number relative to total follicle number. Pigmentation efficiency also decreased with Formulation A with statistical significance at days 1, 3 (FIG. 3—the error bar denotes S.E.M. (Standard Error of the Mean)). The Unpaired two sample Student's T-test was used to determine the significance of the differences between the group (SPSS version 20.0).

### Example 4: Pigmentation Assay with Formula B

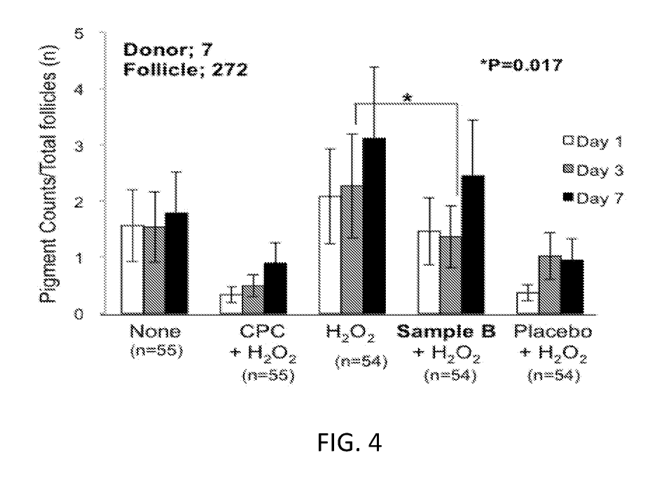

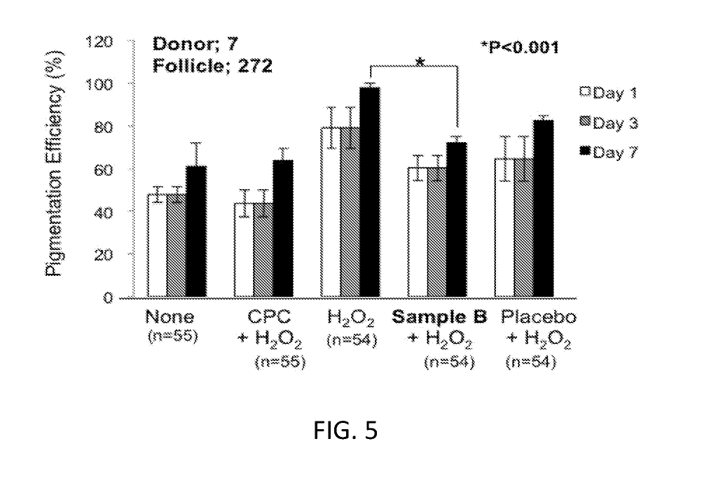

A total of 272 follicles from 7 different donors were used to validate formulation B in the assay of Example 2. H₂O₂ treatment (3 percent) increased pigmentation at the bulge area of the follicle with statistical significance. Positive Control (CPC) was effective on the pigmentation prevention with statistical significance. Pretreatment with Formulation B showed decreased pigmented cell counts at days 1, 3, and 7 compared to the H₂O₂ treatment (FIG. 4). Pigmentation efficiency decreased with Formulation B application with statistical significance at the day 7 of topical applications (FIG. 5). The error bar denotes S.E.M.

### Example 5: Pigmentation Assay with Formula C

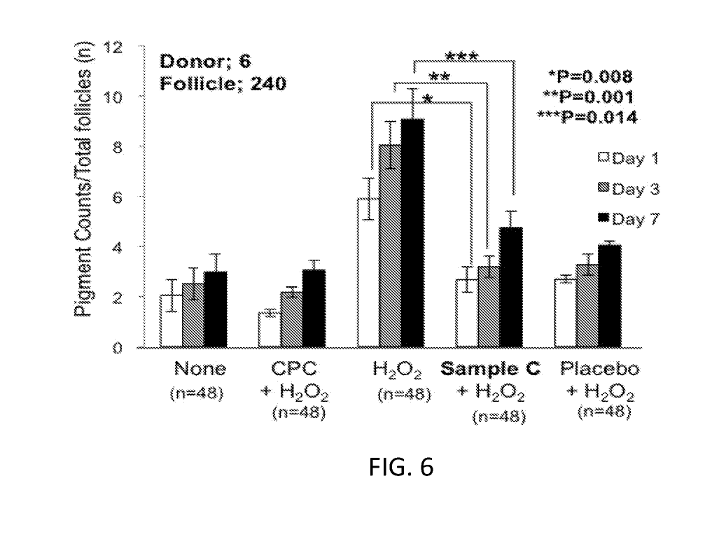

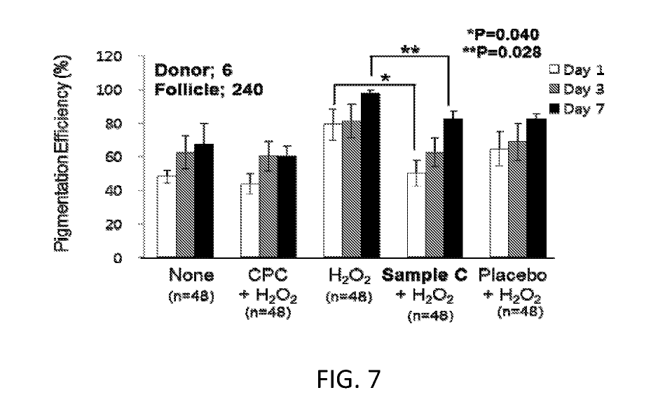

A total of 160 follicles from 6 different donors were used to validate formulation C in the assay of Example 2. H₂O₂ treatment (3 percent) increased pigmentation at the bulge area of the follicle with statistical significance. The Positive Control (CPC) was effective on the pigmentation prevention with statistical significance. Pretreatment with formulation C showed decreased pigmented cell counts at the day 1, 3, 7 of H₂O₂ treatment (FIG. 6). Pigmentation efficiency decreased with Formulation C application with statistical significance at the day 1 and 7 after H₂O₂ treatment (FIG. 7). The error bar denotes S.E.M.

### Example 6: Pigmentation Assay with Formula D

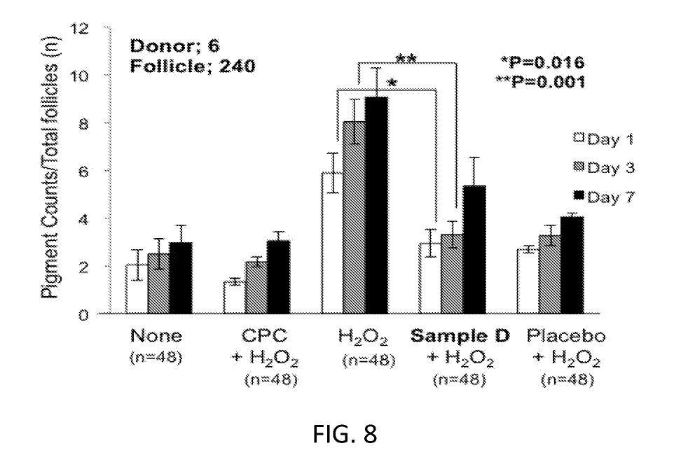

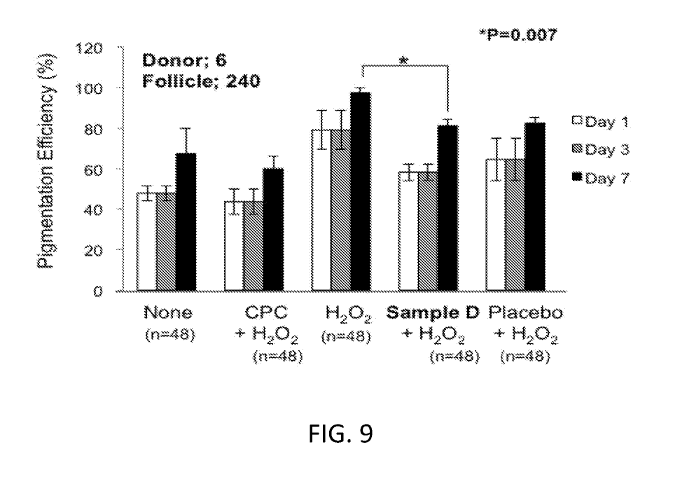

A total of 160 follicles from 6 different donors were used to validate Formulation D in the assay of Example 2. H₂O₂ treatment (3 percent) increased pigmentation at the bulge area of the follicle with statistical significance. The Positive Control (CPC) was effective on the pigmentation prevention with statistical significance. Pretreatment with Formulation D showed decreased pigmented cell counts at the day 1, 3 of H₂O₂ treatment with statistical significance (FIG. 8). Pigmentation efficiency decreased with Formulation D application with statistical significance only at day 7 after H₂O₂ treatment (FIG. 9). The error bar denotes S.E.M.

### Example 7: Microscopic Imaging of Melanoblasts

As indicated by a melanocyte stem cell damage assay, Formulations A and C were the most capable of preventing melanocyte stem cell damage and differentiation into pigmented cells. Both pigmented cell counts and pigmentation efficiency have been shown to be effective with Formulations A and C with statistically significant p-values. With Formulation A, pigmented cell counts were decreased with statistical significance at days 1, 3, and 7. The data showed that Formulations B and D were efficient on days 3 or 7, which implies that these formulations may be slower acting on the follicles or may have delivery problems.

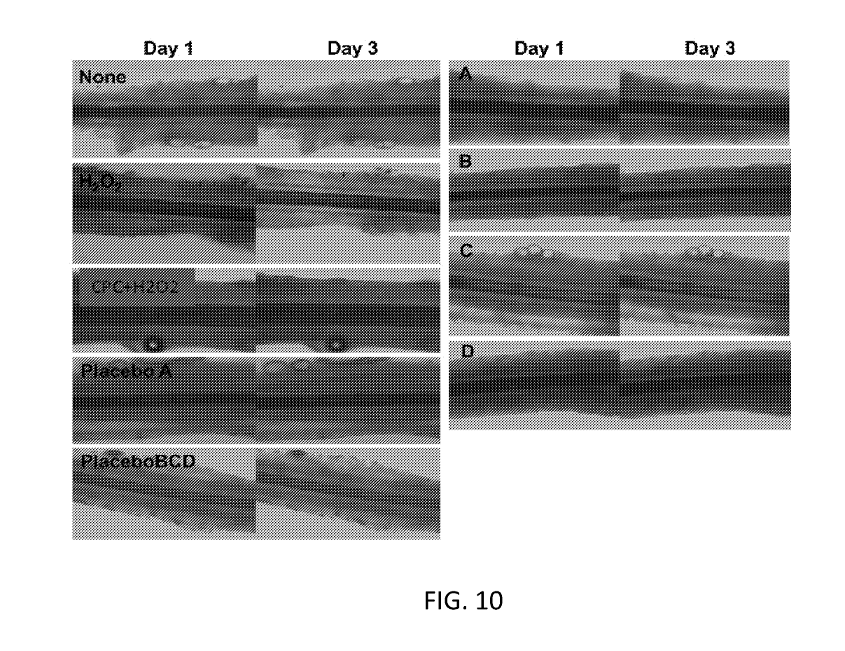

The ectopic pigmentation after application of Formulations A, B, C, and D at the bulge area of the follicles after H₂O₂ damage can be seen in FIG. 10.

### Example 8: Antioxidant Effect of NAC

A Reactive Oxygen Species (ROS) assay was used for individual follicle observation with 5-(and-6)-chloromethyl-2′,7′-dichlorodihydrofluorescein diacetate, acetyl ester (CM-H2DCFDA). CM-H2DCFDA is a chloromethyl derivative of monochlorobimane, 2′,7′-dichlorohydrofluorescein diacetate (H2DCFDA), and is useful as an indicator for general ROS in cells. ROS should be labeled after 2 hours and imaged in 20 minutes, and the protocol included dissection of the follicles first before H₂O₂ treatment. After 2 hours of incubation, ROS were labeled with CM-H2DCFDA for 20 minutes and the follicles were observed with a fluorescent microscopy.

The ROS detection protocol was as follows:

(1) tissue preparation and culture

(2) Isolation of individual follicles

(3) Vertically embedding in semisolid media

(4) Antioxidant treatment (NAC) on the bulge area of the hair follicles

(5) Incubation for 8 hrs.

(6) Treatment with ionizing radiation (e.g., 8.0 Gray) or H₂O₂

(7) Incubation for 2 hrs.

(8) ROS labeling using aminophenyl fluorescein (APF)

(9) Repositioning and incubation in semisolid agar media

(10) Z-stacking imaging & Analysis

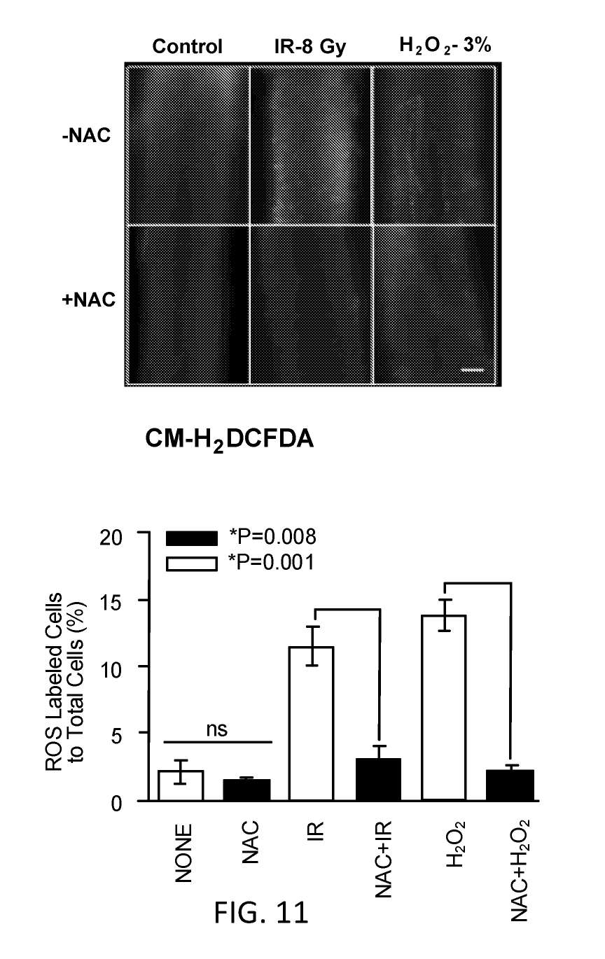

FIG. 11 shows the antioxidant effect of NAC pretreatment. NAC was applied for 24 hours before the ionizing radiation or H₂O₂ treatment and then 2 hours after treatment, ROS labeling and imaging have been done. The ionizing radiation was administered at doses of 4 gy, 8 Gy, or 12 Gy. Topical administration of hydrogen peroxide was used at a dose of 3%. The ROS positive cells were counted and they were normalized to DAPI positive cells. With the NAC treatment, ROS generations were decreased compared to IR or H₂O₂ treatment alone. The result indicates that NAC can be used as the positive control for other test formulations and is very useful on its own in a composition to reduce or inhibit hair graying.

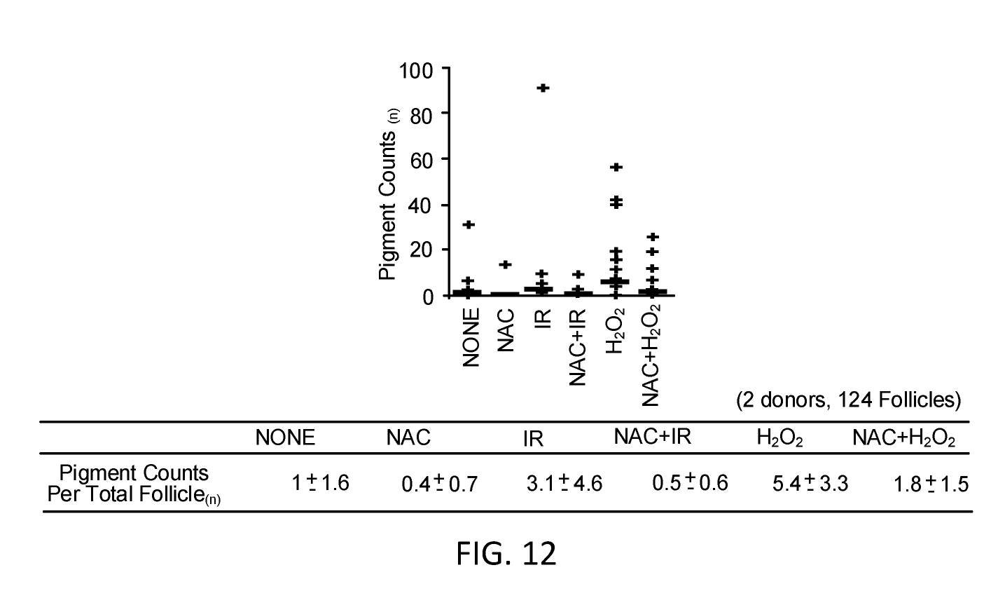

FIG. 12 shows pigmented cell counts after the NAC treatment. Pigmentation efficiency was not high enough to evaluate the statistical significance, but there was a tendency showing decreased pigmented cell counts after the NAC treatment.

### Example 9: Use of Melanocyte Stem Cell Damage and Pigmentation Assay to Measure the Effects of Formulation A and N-Acetyl-Cysteine (NAC) on Hair Graying

Formulation A, placebo A, or the positive control (CPC) were topically applied to the human skin specimens for 24 hours. The specimens were next treated with 3% hydrogen peroxide (H₂O₂) for 24 hours at 37° C. and incubated in 5% CO₂ to trigger the DNA-damage response. After 24 hours of hydrogen peroxide treatment, all portions of the hair follicles were isolated from stress treated human skin scalp. Upon isolation, the hair follicles were washed gently several times in Phosphate-buffered saline (PBS) and transferred intact into soft agar with fresh growth medium in a cell culture incubator at 37° C. and 5% CO₂.

Hair follicles were examined under bright field microscopy after completion of the hydrogen peroxide treatment daily from day 0 (completion of peroxide treatment) to day 7. Pigmented melanocyte stem cells (MSCs) found in the bulge areas of hair follicles were counted in the bright field view.

Formulation A and Placebo A, and the positive control (CPC) were tested, and eight follicles were investigated from each treatment. Paired T-test was applied for data analysis. P-value clearly indicated that hydrogen peroxide-induced pigmentation was suppressed by the CPC. P-value of 0.05 was set as the threshold of statistical significance.

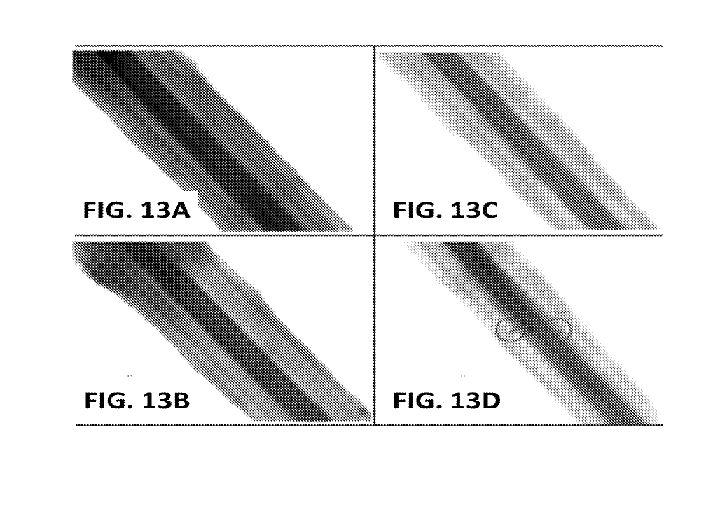

The data showed that this novel drug screening platform can be efficiently used for anti-hair graying drug screening. As shown in FIG. 13A and FIG. 1, individual human hair follicles can be maintained within soft-agar culture for 7 days without significant effects on morphology or changes in pigmentation of bulge cells. In contrast, following exposure to oxidative stress (3% H₂O₂) pigmented bulge melanocytes appeared (FIG. 13D, circled in black). The development of this assay required a significant degree of troubleshooting and optimization to process, culture, and maintain human hair follicles in a healthy state for 7 days, and also to utilize oxidative damage to produce bulge pigmentation, followed by bright field microscopy and quantification.

Pigmented melanoblasts in bulge areas were examined under microscopy in the bright field view before (FIGS. 13A & 13C) and after 7 days culture (FIGS. 13B & 13D) without genotoxic stress (FIG. 13A & FIG. 13B) or with genotoxic stress (3% H₂O₂ in FIGS. 13C & 13D). Pigments can be found circled in FIG. 13D.

Anti-hair graying assays were performed topically in parallel with Formulation A and its placebo, Placebo A, a blank control without hydrogen peroxide (H₂O₂) treatment, hydrogen peroxide treatment, and hydrogen peroxide with a positive control (CPC).

Eight follicles were analyzed in each test set. Formulation A showed a statistically significant protection effect (P-value is 0.007, better than 0.05 significance threshold) on melanocyte stem cell damage and differentiation into pigmented cells.

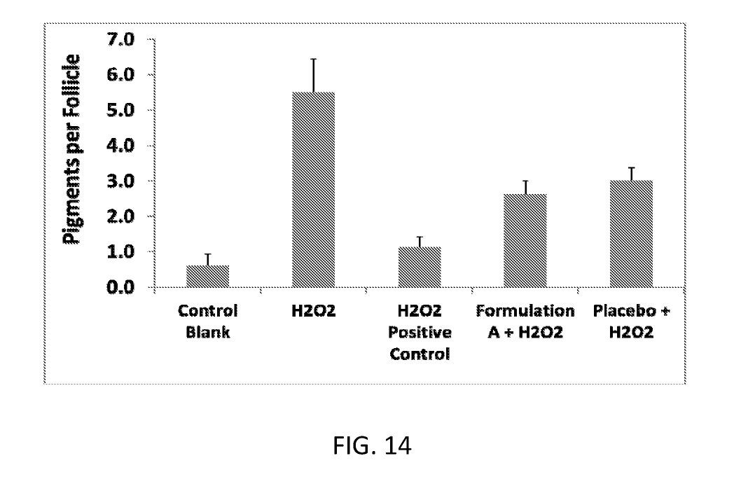

As seen in FIG. 14, a T-test was applied for data analysis. The error bar denotes S.E.M. (the Standard Error of the Mean). Topically applied Formulation A showed statistically significant protection (P-value is 0.007, better than the 0.05 significance threshold) on melanocyte stem cell damage and differentiation into pigmented cells.

In these Examples, the melanocyte stem cell damage and pigmentation assay was used to examine bioactivity of potential anti-hair graying agents by testing human follicle in intact human skin responses, and comparing to positive control activity that was developed in our laboratory. With the result of this melanocyte stem cell damage assay, we could observe that topical application of Formulation A on intact human scalp skin is capable of preventing hair follicle melanocyte stem cell damage and differentiation into pigmented cells, which is consistent with our previous reported findings with treatments of isolated hair follicles.

These studies have utilized a human hair follicle based assay to demonstrate melanocyte stem cell protective activities for the formulations described herein. These data support the application of such agents for purposes of prevention of human hair graying.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method for reducing or inhibiting the graying of hair, the method comprising topically applying a cosmetically effective amount of a formulation to hair follicles and/or to the skin overlying hair follicles one or more times per day, wherein the formulation comprises a combination of a green tea extract; one or more of the group consisting of aroeira (Schinus terebinthifolius) extract, coffee extract, vitamin E, grape seed oligomeric proanthocyanidins (OPC), cocoa extract, Castanea sativa seed extract, hydrolyzed Candida saitoana extract and soybean protein; and one or more cosmetically acceptable carriers comprising an aqueous gel, alcoholic gel, ointment, oil, alcoholic or aqueous fluid, water-in-oil emulsion, oil-in-water emulsion, and a water-in-silicone emulsion.

2. The method of claim 1, wherein the formulation further comprises one or more cosmetically acceptable accessory ingredients.

3. The method of claim 2, wherein the cosmetically acceptable accessory ingredients are selected from the group consisting of xanthan gum, glycerine, EDTA, sodium benzoate, phenoxyethanol, 2-hydroxy fatty alcohol alkoxylate, sodium polyacrylate, polysorbate 20, BHT, disaccharidic gums, ethylhexylglycerin, carbomer, butyleneglycol, acrylate polymers, PEG-40 hydrogenated castor oil, methylisothiazolinone, methylchloroisothiazolinone, propylene glycol, potassium sorbate, polyglyceryl caprylate, fragrance and water.

4. The method of claim 1, wherein the combination comprises between about 0.1% and 15% of the formulation.

5. The method of claim 1, wherein the green tea extract comprises about 0.001% to about 0.5% of the formulation.

6. The method of claim 1, wherein aroeira (Schinus terebinthifolius) extract is included and the aroeira (Schinus terebinthifolius) extract comprises about 0.001% to about 2.5% of the formulation.

7. The method of claim 1, wherein coffee extract is included and the coffee extract comprises about 0.01% to about 1% of the formulation.

8. The method of claim 1, wherein vitamin E is included and the vitamin E comprises about 0.01% to about 3% of the formulation.

9. The method of claim 1, wherein grape seed oligomeric proanthocyanidins (OPC) is included and the grape seed oligomeric proanthocyanidins (OPC) comprises about 0.01% to about 1% of the formulation.

10. The method of claim 1, wherein cocoa extract is included and the cocoa extract comprises about 0.01% to about 1% of the formulation.

11. The method of claim 1, wherein Castanea sativa seed extract is included and the Castanea sativa seed extract comprises about 0.2% to about 5% of the formulation.

12. The method of claim 1, wherein hydrolyzed Candida saitoana extract is included and the hydrolyzed Candida saitoana extract comprises about 0.08% to about 3% of the formulation.

13. The method of claim 1, wherein soybean protein is included and the soybean protein comprises about 0.5% to about 15% of the formulation.

14. The method of claim 3, wherein:
xanthan gum is included and the xanthan gum comprises about 0.001% to about 5% of the formulation;
glycerin is included and the glycerin comprises about 0.001% to about 5% of the formulation;
EDTA is included and the EDTA comprises about 0.001% to about 5% of the formulation;
sodium benzoate is included and the sodium benzoate comprises about 0.001% to about 5% of the formulation;
phenoxyethanol is included and the phenoxyethanol comprises about 0.001% to about 5% of the formulation;
2-hydroxy fatty alcohol alkoxylate is included and the 2-hydroxy fatty alcohol alkoxylate comprises about 0.001% to about 5% of the formulation;
sodium polyacrylate is included and the sodium polyacrylate comprises about 0.001% to about 5% of the formulation;
polysorbate 20 is included and the polysorbate 20 comprises about 0.001% to about 5% of the formulation; and/or
BHT is included and the BHT comprises about 0.001% to about 5% of the formulation.

15. (canceled)

16. (canceled)

17. (canceled)

18. (canceled)

19. (canceled)

20. (canceled)

21. (canceled)

22. (canceled)

23. The method of claim 3, wherein
fragrance is included and the fragrance comprises about 0.001% to about 5% of the formulation.

24. The method of claim 3, wherein
water is included and the water comprises about 0.001% to about 5% of the formulation.

25. The method of claim 1, wherein the formulation comprises about 0.1% of green tea extract, about 0.25% of aroeira (Schinus terebinthifolius) extract and about 0.1% of grape seed oligomeric proanthocyanidins (OPC).

26. The method of claim 1, wherein the formulation comprises about 2.2% of Castanea sativa seed extract, about 1.65% of hydrolyzed Candida saitoana extract, about 0.05% of cocoa extract and about 5.5% soy protein.

27. The method of claim 1, wherein the formulation further comprises N-acetyl-cysteine.

28. The method of claim 1, wherein the formulation reduces oxidative cell stress and/or inhibits melanocyte stem cell depletion.

29. (canceled)
